(19) United States
(12) Patent Application Publication
(10) Pub. No.: US 2025/0050107 A1
(43) Pub. Date: Feb. 13, 2025

(54) METHODS AND SYSTEMS FOR CONTROL AND MODIFICATION OF STIMULATION

(71) Applicant: Boston Scientific Neuromodulation Corporation, Valencia, CA (US)

(72) Inventors: Lisa Denise Moore, Glendale, CA (US); Peter J. Yoo, Burbank, CA (US)

(21) Appl. No.: 18/797,122

(22) Filed: Aug. 7, 2024

Related U.S. Application Data
(60) Provisional application No. 63/532,296, filed on Aug. 11, 2023.

(51) Int. Cl. A61N 1/36

(52) U.S. Cl. CPC A61N 1/36132; A61N 1/36157

(57) ABSTRACT

Electrical stimulation systems and methods for operation and management of the electrical stimulation system are described. One method for operation of an electrical stimulation system includes receiving a request to modify stimulation; displaying a graph for at least one adjustable stimulation parameter; indicating on the graph a current setting of each of the at least one adjustable stimulation parameter and a range around the current setting of at least one of the at least one adjustable stimulation parameter which represents at least one of a minor change in the stimulation based on a predefined minor change criteria or a moderate change in the stimulation based on a predefined moderate change criteria; receiving a selection of a new setting for at least one of the at least one adjustable stimulation parameter; and modifying the stimulation according to the selection.

### CROSS-REFERENCE TO RELATED APPLICATIONS

This application claims the benefit under 35 U.S.C. § 119(e) of U.S. Provisional Patent Application Ser. No. 63/532,296, filed Aug. 11, 2023, which is incorporated herein by reference.

### FIELD

The present disclosure is directed to the area of therapeutic stimulation systems and methods of making and using the systems. The present disclosure is also directed to methods and systems for control and modification of therapeutic stimulation including control and modification by a patient or non-clinician caregiver.

### BACKGROUND

Implantable electrical and optical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Deep brain stimulation can be used to treat a variety of diseases and disorders.

Stimulators have been developed to provide therapy for a variety of treatments. A stimulator can include a control module (with a pulse generator) and one or more stimulator electrodes or optical emitter. The one or more stimulator electrodes or optical emitters can be disposed along one or more leads, or along the control module, or both. In electrical stimulation systems, stimulator electrodes are in contact with or near the nerves, muscles, or other tissue to be stimulated. The pulse generator in the control module generates electrical pulses that are delivered by the electrodes to body tissue.

### BRIEF SUMMARY

One aspect is a method for operation of a stimulation system. The method includes receiving a request to modify stimulation; displaying a graph for at least one adjustable stimulation parameter; indicating on the graph a current setting of each of the at least one adjustable stimulation parameter and a range around the current setting of at least one of the at least one adjustable stimulation parameter which represents at least one of a minor change in the stimulation based on a predefined minor change criteria or a moderate change in the stimulation based on a predefined moderate change criteria; receiving a selection of a new setting for at least one of the at least one adjustable stimulation parameter; and modifying the stimulation according to the selection.

Another aspect is a stimulation system having an implantable control module and a programmer for programming the implantable control module, the programmer including a memory having instructions stored thereon and a processor coupled to the memory and configured to execute the instructions to perform actions. The actions include receiving a request to modify stimulation; displaying a graph for at least one adjustable stimulation parameter; indicating on the graph a current setting of each of the at least one adjustable stimulation parameter and a range around the current setting of at least one of the at least one adjustable stimulation parameter which represents at least one of a minor change in the stimulation based on a predefined minor change criteria or a moderate change in the stimulation based on a predefined moderate change criteria; receiving a selection of a new setting for at least one of the at least one adjustable stimulation parameter; and modifying the stimulation according to the selection.

A further aspect is a non-transitory computer readable memory having instructions stored thereon for operation of a stimulation system, wherein the instructions, when executed by a processor, perform actions. The actions include receiving a request to modify stimulation; displaying a graph for at least one adjustable stimulation parameter; indicating on the graph a current setting of each of the at least one adjustable stimulation parameter and a range around the current setting of at least one of the at least one adjustable stimulation parameter which represents at least one of a minor change in the stimulation based on a predefined minor change criteria or a moderate change in the stimulation based on a predefined moderate change criteria; receiving a selection of a new setting for at least one of the at least one adjustable stimulation parameter; and modifying the stimulation according to the selection.

In at least some embodiments, the indicating includes indicating on the graph both the range representing the minor change and the range representing the moderate change. In at least some embodiments, the indicating includes indicating on the graph at least one previous setting for at least one of the at least one adjustable stimulation parameter. In at least some embodiments, for at least one of the at least one previous setting, the indication signifies an assessment of the stimulation provided by the previous setting. In at least some embodiments, the indicating further includes indicating on the graph an estimated disliked range based on a one of the at least one previous setting that has been assessed as providing disliked stimulation. In at least some embodiments, the method further includes displaying a medication state for either the current setting or at least one of the at least one previous setting.

In at least some embodiments, the method or actions further include querying whether the modified stimulation is acceptable after providing the modified stimulation for a predefined period of time and continuing the modified stimulation when a response to the query indicates that the modified stimulation is acceptable. In at least some embodiments, the method or actions further include halting the modified stimulation when the response to the query indicates that the modified stimulation is not acceptable. In at least some embodiments, the method or actions further include returning to the previous stimulation when the response to the query indicates that the modified stimulation is not acceptable.

Yet another aspect is a method for operation of a stimulation system. The method includes receiving, from a patient or a non-clinician caregiver, at least one priority for stimulation of the patient, wherein at least one of the at least one priority is selected from an improvement in a symptom or a set of symptoms, a reduction in a side effect or a set of side effects, an improvement in a therapeutic effect or a set of therapeutic effects, avoidance of a side effect or a set of side effects, a reduction in battery use, or any combination thereof; identifying and providing at least one stimulation program based on the at least one priority; receiving a selection of a one of the at least one program; and stimulating the patient using the selected one of the at least one stimulation program.

Another aspect is a stimulation system having an implantable control module configured for implantation in a patient and a programmer for programming the implantable control module, the programmer including a memory having instructions stored thereon and a processor coupled to the memory and configured to execute the instructions to perform actions. The actions include receiving, from a patient or a non-clinician caregiver, at least one priority for stimulation of the patient, wherein at least one of the at least one priority is selected from an improvement in a symptom or a set of symptoms, a reduction in a side effect or a set of side effects, an improvement in a therapeutic effect or a set of therapeutic effects, avoidance of a side effect or a set of side effects, a reduction in battery use, or any combination thereof; identifying and providing at least one program based on the at least one priority; receiving a selection of a one of the at least one stimulation program; and stimulating the patient using the selected one of the at least one stimulation program.

A further aspect is a non-transitory computer readable memory having instructions stored thereon for operation of a stimulation system, wherein the instructions, when executed by a processor, perform actions. The actions include receiving, from a patient or a non-clinician caregiver, at least one priority for stimulation of the patient, wherein at least one of the at least one priority is selected from an improvement in a symptom or a set of symptoms, a reduction in a side effect or a set of side effects, an improvement in a therapeutic effect or a set of therapeutic effects, avoidance of a side effect or a set of side effects, a reduction in battery use, or any combination thereof; identifying and providing at least one program based on the at least one priority; receiving a selection of a one of the at least one stimulation program; and stimulating the patient using the selected one of the at least one stimulation program.

In at least some embodiments, the receiving includes ranking a plurality of the priorities. In at least some embodiments, the identifying and providing includes identifying and providing the at least one stimulation program based on the ranking of the plurality of priorities.

In at least some embodiments, the method or the actions further include querying whether the selected one of the at least one stimulation program is acceptable after providing the selected one of the at least one stimulation program for a predefined period of time and continuing the selected one of the at least one stimulation program when a response to the query indicates that the selected one of the at least one stimulation program is acceptable. In at least some embodiments, the method or the actions further include, when the response to the query indicates that the selected one of the at least one stimulation program is not acceptable, either halting the selected one of the at least one stimulation program or returning to a previous stimulation program.

One aspect is a method for operation of a stimulation management system. The method includes obtaining, from at least one sensor, data for the patient regarding at least one symptom, at least one therapeutic effect, at least one side effect, or any combination thereof; connecting the at least one sensor to a processing device; delivering the data to the processing device; processing the data to determine at least one score based on the data; determining an adjustment to stimulation of the patient based on the at least one score; and stimulating the patient according to the adjustment.

In at least some embodiments, the connecting includes attaching, or coupling with a cable, at least one of the at least one sensor to the processing device. In at least some embodiments, the method further includes displaying the at least one score for the patient or a non-clinician caregiver. In at least some embodiments, the method further includes providing a stimulation report.

In at least some embodiments, the method further includes querying whether the adjustment to the stimulation is acceptable after providing the adjustment for a predefined period of time and continuing the adjustment when a response to the query indicates that the adjustment is acceptable. In at least some embodiments, the method further includes halting the stimulation when the response to the query indicates that the adjustment is not acceptable. In at least some embodiments, the method further includes returning to a previous stimulation when the response to the query indicates that the adjustment is not acceptable.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system;

FIG. 2 is a schematic side view of one embodiment of an electrical stimulation lead;

FIG. 3 is a schematic block diagram of a system for practicing the methods described herein;

FIG. 4 is a flowchart of one embodiment of a method a method for operation of a stimulation system;

FIG. 5A illustrates one embodiment of graphs of stimulation intensity and tested settings for modification of that stimulation parameter;

FIG. 5B illustrates another embodiment of graphs of stimulation intensity and tested settings for modification of that stimulation parameter;

FIG. 6 is a flowchart of another embodiment of a method for operation of a stimulation system;

FIG. 7 is a block diagram of one embodiment of a stimulation management system; and

FIG. 8 is a flowchart of one embodiment of a method for operating a stimulation management system.

### DETAILED DESCRIPTION

The present disclosure is directed to the area of therapeutic stimulation systems and methods of making and using the systems. The present disclosure is also directed to methods and systems for control and modification of therapeutic stimulation including control and modification by a patient or non-clinician caregiver.

Implantable electrical stimulation systems and devices are used herein to exemplify the inventions, but it will be understood that these inventions can be utilized with other stimulation or modulation systems and devices, such as optical or electrical/optical stimulation or modulation systems. Examples of implantable electrical stimulation systems include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties. Examples of optical stimulation or modulation systems or electrical/optical stimulation systems, which include one or more optical emitters in addition to, or as an alternative to, electrodes, are found in U.S. Pat. Nos. 9,415,154; 10,335,607; 10,625,072; and 10,814,140 and U.S. Patent Application Publications Nos. 2013/0317572; 2013/0317573; 2017/0259078; 2017/0225007; 2018/0110971; 2018/0369606; 2018/0369607; 2019/0209849; 2019/0209834; 2020/0094047; 2020/0155584; 2020/0376262; 2021/0008388; 2021/0008389; 2021/0016111; and 2022/0072329, all of which are incorporated by reference in their entireties.

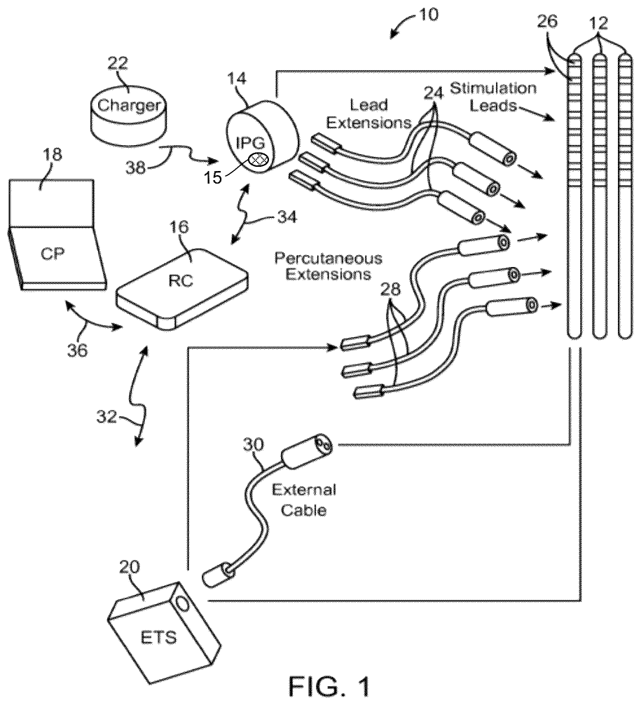

Turning to FIG. 1, one embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG and ETS are examples of control modules for the electrical stimulation system.

The IPG 14 is physically connected, optionally via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The implantable pulse generator can be implanted into a patient's body, for example, below the patient's clavicle area or within the patient's buttocks or abdominal cavity or at any other suitable site. The implantable pulse generator can have multiple stimulation channels which may be independently programmable to control the magnitude of the current stimulus from each channel. In some embodiments, the implantable pulse generator can have any suitable number of stimulation channels including, but not limited to, 4, 6, 8, 12, 16, 32, or more stimulation channels. The implantable pulse generator can have one, two, three, four, or more connector ports, for receiving the terminals of the leads and/or lead extensions.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters (e.g., a stimulation program). One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets (e.g., different stimulation programs). The IPG 14 may also be operated to modify the programmed stimulation parameters (e.g., a stimulation program) to actively control the characteristics of the electrical stimulation energy output by the IPG 14. The CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14. In at least some embodiments, the RC 16 can be a mobile phone, tablet, desktop computer, or the like.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters (e.g., a stimulation program) can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Examples of electrical stimulation systems can be found at U.S. Pat. Nos. 6,181,969; 6,516,227; 6,609,029; 6,609,032; 6,741,892; 6,895,280; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated herein by reference in their entireties.

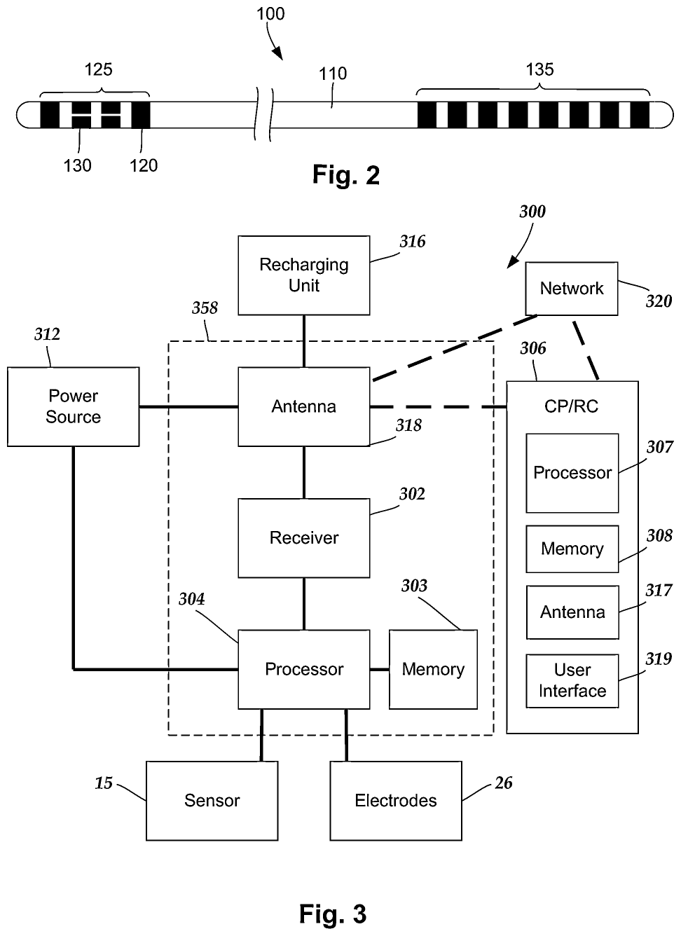

FIG. 2 illustrates one embodiment of a lead 100 with electrodes 125 disposed at least partially about a circumference of the lead 100 along a distal end portion of the lead 100 and terminals 135 disposed along a proximal end portion of the lead 100.

The lead 100 can be implanted near or within the desired portion of the body to be stimulated such as, for example, the brain, spinal cord, or other body organs or tissues. In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 100 can be inserted into the cranium and brain tissue with the assistance of a stylet (not shown). The lead 100 can be guided to the target location within the brain using, for example, a stereotactic frame and a microdrive motor system. In at least some embodiments, the microdrive motor system can be fully or partially automatic. The microdrive motor system may be configured to perform at least one of the following actions (alone or in combination): insert the lead 100, advance the lead 100, retract the lead 100, or rotate the lead 100.

The lead 100 for electrical stimulation can include stimulation electrodes, recording electrodes, or both. In at least some embodiments, the lead 100 is rotatable so that the stimulation electrodes can be aligned with the target neurons after the neurons have been located using the recording electrodes.

Stimulation electrodes may be disposed on the circumference of the lead 100 to stimulate the target neurons, neural elements, or other tissue. Stimulation electrodes may be ring shaped so that current projects from each electrode radially from the position of the electrode along a length of the lead 100. In the embodiment of FIG. 2, two of the electrodes 125 are ring electrodes 120. Ring electrodes typically do not enable stimulus current to be directed from only a limited angular range around a lead. Segmented electrodes 130, however, can be used to direct stimulus current to a selected angular range around a lead. When segmented electrodes are used in conjunction with an implantable pulse generator that delivers constant current stimulus, current steering can be achieved to deliver the stimulus more precisely to a position around an axis of a lead (i.e., radial positioning around the axis of a lead). To achieve current steering, segmented electrodes can be utilized in addition to, or as an alternative to, ring electrodes.

The lead 100 includes a lead body 110, terminals 135, at least one ring electrode 120, and at least one set of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethane-urea, polyethylene, or the like. Once implanted in the body, the lead 100 may be in contact with body tissue for extended periods of time. In at least some embodiments, the lead 100 has a cross-sectional diameter of no more than 1.5 mm and may be in the range of 0.5 to 1.5 mm. In at least some embodiments, the lead 100 has a length of at least 10 cm and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes 125 are made of a material that is biocompatible and does not substantially corrode under expected operating conditions in the operating environment for the expected duration of use.

Each of the electrodes 125 can either be used or unused (OFF). When an electrode is used, the electrode can be used as an anode or cathode and carry anodic or cathodic current. In some instances, an electrode might be an anode for a period of time and a cathode for a period of time.

Deep brain stimulation leads may include at least one set of segmented electrodes. Segmented electrodes may provide for superior current steering than ring electrodes because target structures in deep brain stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue. Examples of leads with segmented electrodes include U.S. Pat. Nos. 8,473,061; 8,571,665; 8,792,993; 9,248,272; 9,775,988; and 10,286,205; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; and 2015/0051681, all of which are incorporated herein by reference.

FIG. 3 is a schematic overview of one embodiment of components of an electrical stimulation system 300 including an electronic subassembly 310 disposed within an IPG 14 (FIG. 1). It will be understood that the electrical stimulation system can include more, fewer, or different components and can have a variety of different configurations including those configurations disclosed in the stimulator references cited herein.

The IPG 14 (FIG. 1) can include, for example, a power source 312, antenna 318, receiver 302, processor 304, and memory 305, as well as a sensor 15 that can be disposed in, or on, the IPG. Some of the components (for example, power source 312, antenna 318, receiver 302, processor 304, and memory 305) of the electrical stimulation system can be positioned on one or more circuit boards or similar carriers within a sealed housing of the IPG 14 (FIG. 1), if desired. Unless indicated otherwise, the term “processor” refers to both embodiments with a single processor and embodiments with multiple processors.

An external device, such as a CP or RC 306, can include a processor 307, memory 308, an antenna 317, and a user interface 319. The user interface 319 can include, but is not limited to, a display screen on which a digital user interface can be displayed and any suitable user input device, such as a keyboard, touchscreen, mouse, track ball, or the like or any combination thereof.

Any power source 312 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference in its entirety.

As another alternative, power can be supplied by an external power source through inductive coupling via the antenna 318 or a secondary antenna. The external power source can be in a device that is mounted on the skin of the user or in a unit that is provided near the user on a permanent or periodic basis.

If the power source 312 is a rechargeable battery, the battery may be recharged using the antenna 318, if desired. Power can be provided to the battery for recharging by inductively coupling the battery through the antenna to a recharging unit 316 external to the user. Examples of such arrangements can be found in the references identified above.

In one embodiment, electrical current is emitted by the electrodes 26 on the lead body to stimulate nerve fibers, muscle fibers, or other body tissues near the electrical stimulation system. A processor 304 is generally included to control the timing and electrical characteristics of the electrical stimulation system. For example, the processor 304 can, if desired, control one or more of the timing, frequency, amplitude, width, and waveform of the pulses. In addition, the processor 304 can select which electrodes can be used to provide stimulation, if desired. In some embodiments, the processor 304 may select which electrode(s) are cathodes and which electrode(s) are anodes. In some embodiments, the processor 304 may be used to identify which electrodes provide the most useful stimulation of the desired tissue. Instructions for the processor 304 can be stored on the memory 305. Instructions for the processor 307 can be stored on the memory 308.

Any processor 304 can be used for the IPG and can be as simple as an electronic device that, for example, produces pulses at a regular interval or the processor can be capable of receiving and interpreting instructions from the CP/RC 306 (such as CP 18 or RC 16 of FIG. 1) that, for example, allows modification of pulse characteristics. In the illustrated embodiment, the processor 304 is coupled to a receiver 302 which, in turn, is coupled to the antenna 318. This allows the processor 304 to receive instructions from an external source to, for example, direct the pulse characteristics and the selection of electrodes, if desired. Any suitable processor 307 can be used for the CP/RC 306.

Any suitable memory 305, 308 can be used including computer-readable storage media may include, but is not limited to, volatile, nonvolatile, non-transitory, removable, and non-removable media implemented in any method or technology for storage of information, such as computer readable instructions, data structures, program modules, or other data. Examples of computer-readable storage media include, but are not limited to, RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a processor.

In one embodiment, the antenna 318 is capable of receiving signals (e.g., RF signals) from an antenna 317 of a CP/RC 306 (see, CP 18 or RC 16 of FIG. 1) which is programmed or otherwise operated by a user. The signals sent to the processor 304 via the antenna 318 and receiver 302 can be used to modify or otherwise direct the operation of the electrical stimulation system. For example, the signals may be used to modify the pulses of the electrical stimulation system such as modifying one or more of pulse width, pulse frequency, pulse waveform, and pulse amplitude. The signals may also direct the electrical stimulation system 300 to cease operation, to start operation, to start signal acquisition, to stop signal acquisition, to start charging the battery, or to stop charging the battery. In other embodiments, the stimulation system does not include an antenna 318 or receiver 302 and the processor 304 operates as programmed.

Optionally, the electrical stimulation system 300 may include a transmitter (not shown) coupled to the processor 304 and the antenna 318 for transmitting signals back to the CP/RC 306 or another unit capable of receiving the signals. For example, the electrical stimulation system 300 may transmit signals indicating whether the electrical stimulation system 300 is operating properly or not or indicating when the battery needs to be charged or the level of charge remaining in the battery. The processor 304 may also be capable of transmitting information about the pulse characteristics so that a user or clinician can determine or verify the characteristics.

Transmission of signals can occur using any suitable method, technique, or platform including, but not limited to, inductive transmission, radiofrequency transmission, Bluetooth™, Wi-Fi, cellular transmission, near field transmission, infrared transmission, or the like or any combination thereof. In addition, the IPG 14 can be wirelessly coupled to the RC 16 or CP 18 using any suitable arrangement include direct transmission or transmission through a network, such as a local area network, wide area network, the Internet, or the like or any combination thereof. The CP 18 or RC 16 may also be capable of coupling to, and sending data or other information to, a network 320, such as a local area network, wide area network, the Internet, or the like or any combination thereof.

As described above, a clinician can program a stimulation system to provide stimulation to a patient. It may also be desirable to allow the patient, other non-clinician caregivers, or the like to modify the stimulation between visits with the clinician. In at least some embodiments, the methods and systems described below are provided for use by a patient, a non-clinician caregiver, or any other individual that is not a medical professional or not a medical professional that is professionally involved in providing stimulation therapy to the patient. The term “patient or caregiver” will be used herein to represent these individuals. The “adjust” and other forms of the word “adjust” (e.g., adjustment) are respectively synonymous to the terms “modify” and corresponding forms of the word “modify” (e.g., modification).

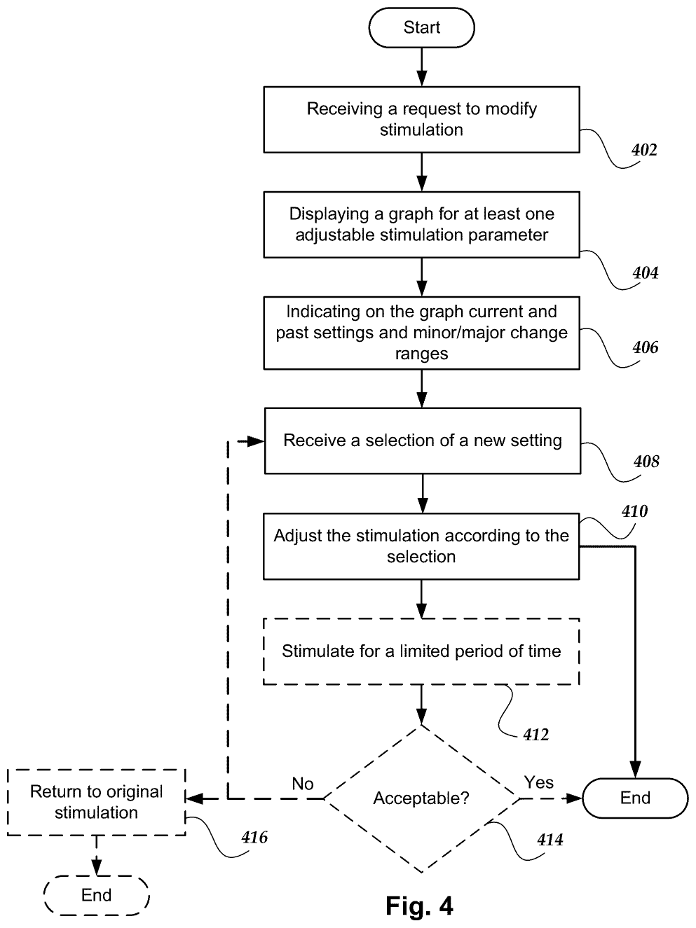

A programmer, such as a RC 16 or CP 18, can be used to modify stimulation. In at least some instances, a patient or caregiver may modify stimulation using such a programmer. FIG. 4 illustrates one embodiment of a method for operation of an electrical stimulation system that includes an implantable control module configured for implantation in a patient and a lead coupled, or coupleable, to the implantable control module and having electrodes disposed along a distal portion of the lead.

In step 402, a request is received to modify the stimulation. In at least some embodiments, a patient or caregiver sends a request to a programmer, such a RC 16 or CP 18, to modify the stimulation. In at least some embodiments, the request may be in the form of a request for improvement in one or more symptoms, a reduction in one or more side effects, or an improvement in one or more therapeutic effects; a general request for modification; a request to change a particular parameter or parameters; an improvement in an activity that is associated with one or more symptoms or side effects; or the like or any combination thereof.

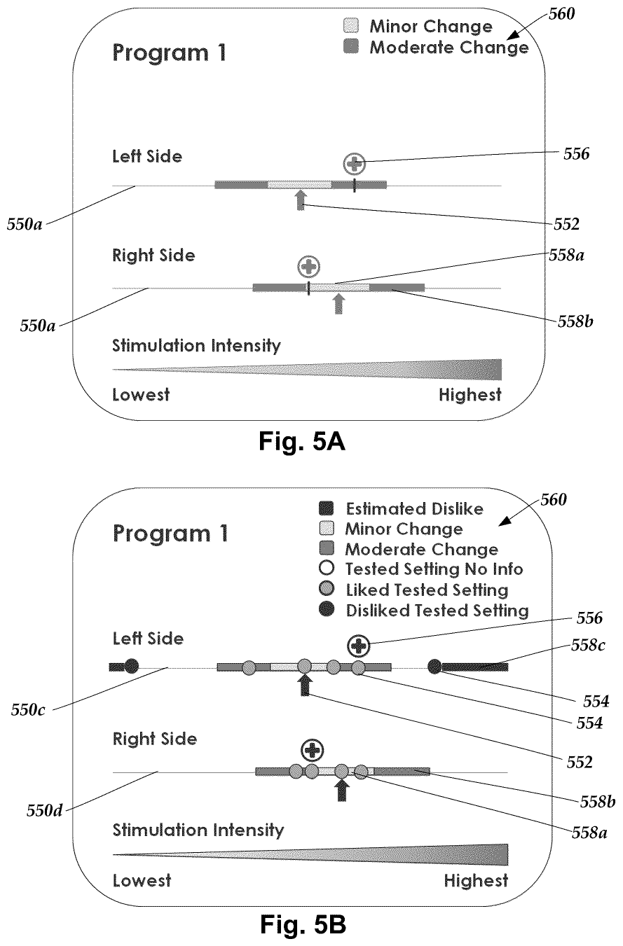

In step 404, at least one graph 550a, 550b, 550c, 550d is displayed for at least one adjustable stimulation parameter, as illustrated in FIGS. 5A and 5B. In at least some embodiments, the graph 550a, 550b, 550c, 550d is a one-dimensional graph, such as the graphs illustrated in FIGS. 5A and 5B, of one adjustable stimulation parameter. Examples of the stimulation parameter include, but are not limited to, stimulation intensity (as illustrated in FIGS. 5A and 5B), stimulation amplitude, electrode selection, electrode fractionalization, stimulation duration, pulse duration, pulse frequency, or the like or any combination thereof. In at least some embodiments, stimulation intensity and stimulation amplitude are synonymous. In at least some other embodiments, stimulation intensity reflects user perception of stimulation and stimulation amplitude is the amplitude of the current or voltage applied.

In other embodiments, the graph can be a two- or three-dimensional graph, such as a clinical effects map. Examples of two- and three-dimensional clinical effects map can be found in U.S. Pat. Nos. 9,227,074; 9,248,296; 9,358,398; 9,474,903; 10,071,249; 10,357,657; 10,369,364; 10,603,498; and 10,716,505; U.S. Patent Application Publications Nos. 2014/0243926; 2014/0276707; 2014/0277282; 2014/0277284; 2018/0264278; 2020/0376263; 2020/0398057; and 2021/0023374; U.S. patent application Ser. No. 18/075,835; and U.S. Provisional Patent Application Ser. Nos. 63/425,149 and 63/432,628, all of which are incorporated herein by reference in their entireties. As examples, a two-dimensional graph can include one axis representing stimulation amplitude and another axis representing electrode selection (for example, position of the stimulation along the longitudinal axis of the lead or around the circumference of the lead). As another example, a two-dimensional graph can be a polar graph such as, for example, a graph with the angular coordinate corresponding to electrode selection and the radial coordinate corresponding to stimulation amplitude.

In at least some embodiments, graphs 550a, 550b (FIG. 5A) or 550c, 550d (FIG. 5B) for two or more leads, such as two leads on different sides (e.g., the right side and the left side) of, or positions within, the brain, as illustrated in FIGS. 5A and 5B. Any or all of the graphs 550a, 550b, 550c, 550d can include any of the markings, ranges, and other elements described below.

In step 406, a current setting 552 (indicated by an arrow in FIGS. 5A and 5B) for each of the at least one adjustable stimulation parameter is displayed on the graph 550a, 550b, 550c, 550d, as illustrated in FIGS. 5A and 5B. In at least some embodiments, the graph 550a, 550b, 550c, 550d is displayed on the programmer, such as the RC 16 or CP18. In at least some embodiments, at least one previous setting 554 (indicated by circles in FIG. 5B with darker circles for disliked previous settings and lighter circles for liked/acceptable settings) for at least one of the at least one adjustable stimulation parameter is displayed, as illustrated in FIGS. 5A and 5B. In at least some embodiments, a clinician setting 556 (indicated in FIGS. 5A and 5B by a + in a circle) for each of the at least one adjustable stimulation parameter is displayed on the graph 550a, 550b, 550c, 550d, as illustrated in FIGS. 5A and 5B. Each clinician setting 556 can represent a particular setting programmed by the clinician.

In at least some embodiments, the previous setting(s) 554 or the clinician setting(s) 556 (or both) are indicated on the graph 550a, 550b, 550c, 550d according to an evaluation of the stimulation at the previous setting(s) or clinician setting(s). For example, the previous setting(s) 554 or the clinician setting(s) 556 may be indicated as being liked (for example, the previous settings 554 in FIG. 5B with lighter circles), disliked (for example, the previous settings 554 in FIG. 5B with dark circles), or neutral/no information. Any other equivalent or other descriptive terminology can be used. In at least some embodiments, the indication can include a selection of color, shading, cross-hatching, or the like for a marker on the graph representing the previous setting(s). In at least some embodiments, the graph 550a, 550b, 550c, 550d can include a legend 560 to explain the symbols used in the graph.

In at least some embodiments, the graph 550a, 550b, 550c, 550d may include one or more indications (for example, ranges 558a, 558b, 558c) on the graph representing predicted responses to stimulation at untried settings. Any other suitable method for predicting response to stimulation can be used including, but not limited to using responses to previous setting(s); the estimation techniques described in any of the references cited herein including U.S. Pat. Nos. 8,326,433; 8,675,945; 8,831,731; 8,849,632; 8,958,615; 10,265,528; and 10,603,498; U.S. Patent Application Publications Nos. 2009/0287272; 2009/0287273; 2012/0314924; 2013/0116744; 2014/0122379; 2015/0066111; and 2022/0339448, all of which are incorporated herein by reference in their entireties; or the like or any combination thereof.

In at least some embodiments, the graph 550a, 550b, 550c, 550d indicates a minor change range 558a around the current setting 552 of each of the at least one adjustable stimulation parameter which represents an estimation that a selection within the range will result in a minor change in the stimulation based on at least one minor change criterion. In at least some embodiments, the graph indicates moderate change range 558b around the current setting 552 of each of the at least one adjustable stimulation parameter which represents an estimation that a selection within the range will result in a moderate change in the stimulation based on at least one moderate change criterion. The minor and moderate change criteria can be, for example, an indication of an expected perception by the patient of the stimulation change or an estimated change in the stimulation region (for example, an estimated change in the size of the stimulation region, an estimated change in the size of a target region that is stimulated, an estimated change in a size of a side effect region that is stimulated, or the like or any combination thereof). In at least some embodiments, the minor or moderate change criterion/criteria (or both) are based on predicted response to stimulation for a number of untried setting values. In at least some embodiments, the minor or moderate change criterion/criteria (or both) reflect defined regions of charge density change arising from application of the stimulation.

As an example, stimulation can be provided using common Parkinson's Disease stimulation parameters for standard upper limb movement symptoms, which include a selected electrode fractionalization and an amplitude between 2 and 3.5 mA. In at least some embodiments, where there are no side effects within 1 mA of these stimulation parameters, a minor change is amplitude, with the same fractionalization, may be in the range of 0.1 to 0.3 mA. In at least some embodiments, a minor change corresponds to a change likely to cause no or minimal detectable change to the patient within an acute application of, for example, 30 minutes. In at least some embodiments, a modest change would correspond to an amplitude change of 0.4-0.7 mA. In at least some embodiments, a moderate change would be expected to result in a small, but detectable change, to a patient's symptoms and side effects within an acute application of, for example, 30 minutes.

In at least some embodiments, the minor or moderate change criteria can be derived from predicted changes in stimulation volume. In at least some embodiments, the minor or moderate change criteria may be asymmetric. In at least some embodiments, when a patient is attempting to improve a symptom and there is no side effect nearby, the system is biased towards increasing stimulation as a beneficial solution. In at least some embodiments, when a patient is attempting to address a side effect, the system is biased towards decreasing stimulation. In at least some embodiments, when increasing stimulation to address a symptom, nearby side effects can impact the minor and moderate change criteria, as approaching the side effect is more likely to trigger a side effect then increasing the stimulation in an volume of tissue that has been shown or predicted to be free of side effects. In at least some embodiments, as stimulation parameters approaches a side effect, the minor and moderate change criteria may be condensed such that an increase in amplitude of a smaller magnitude is regarded as a moderate change.

In at least some embodiments, the graph 550a, 550b, 550c, 550d indicates an estimated dislike range 558c. In at least some embodiments, the estimated dislike range 558c is based on previous stimulation setting(s) that were indicated as disliked by the patient, previous patient's experiences, estimated stimulation outcomes, previous stimulation setting(s) that induced or increased one or more side effects or symptoms, or the like or any combination thereof. For example, in FIG. 5B, the range at higher or lower stimulation intensity from a previous disliked setting is indicated as a dislike range 558c as the patient is unlikely to find settings in that range that will be liked in view of the previous disliked setting.

In at least some embodiments, the patient or caregiver is allowed to change one or more adjustable stimulation parameters along a full range of those adjustable stimulation parameters. In at least some embodiments, the patient or caregiver is allowed to change one or more adjustable stimulation parameters along a range of those adjustable stimulation parameters limited by boundaries selected by a clinician or other source. In at least some embodiments, the patient or caregiver is allowed to change one or more adjustable stimulation parameters along a full range of those adjustable stimulation parameters, not only those values determined to be within the range(s) denoted as minor or moderate change ranges 558a, 558b.

In at least some embodiments, if the value(s) of the stimulation parameter(s) selected by the patient or caregiver are outside of ranges denoted as minor or moderate change ranges 558a, 558b, the patient or caregiver may be provided a notification that the changes may result in significant modifications to the stimulation therapy. In at least some embodiments, for any value of a stimulation parameter that the patient or caregiver selects and has been previously used, the patient or caregiver may be provided any previous results or feedback (see below) for the value(s).

In at least some embodiments, additional information can be displayed with the graph 550a, 550b, 550c, 550d or selectable for display with or without the graph. Examples of such information can include, but are not limited to, estimated setting modifications to improve a specific symptom or a set of symptoms or to reduce a specific side effect or a set of side effects; a priority or weighting, by the user, of one or more symptoms; data from previously tested settings; current medication state; timing of last medication; controls to confirm or edit time of last medication; controls to change the time period of information on the display (for example, 1, 2, or 5 days, 1, 2, 3, or 4 weeks, 1, 2, 3, or 6 months, or 1, 2, or more years or any other suitable time period); controls for altering the display or for sorting displayed elements; or the like or any combination thereof.

In step 408, a selection of a new setting (or settings) is received. The selection can be made using any suitable user interface or device including, but not limited to, a touchscreen, a keyboard, a mouse, remote control, voice input, or the like or any combination thereof and may include selection from a menu, a selection on the graph 550a, 550b, 550c, 550d, an input of the new setting, or the like or any combination thereof.

In step 410, the stimulation is adjusted according to the selection. In at least some embodiments, the programmer, such RC 16 or CP 18, sends programming instructions to the IPG 14 or ETS 20. In at least some embodiments, the method ends at step 410.

In at least some other embodiments, in optional step 412, the patient is stimulated for a limited period of time using the new setting (or settings). For example, the stimulation can be applied for 1, 5, 10, 15, 20, 30, 45, or 60 minutes; 1, 2, 4, 6, 8, 12, or 18 hours; or 1, 2, 5, or 7 days or more (or any other suitable time period). In at least some embodiments, the patient or caregiver can halt the stimulation at any time. In at least some embodiments, the patient or caregiver can return the stimulation to the previous setting(s) at any time. In at least some embodiment, the patient or caregiver is queried by the programmer, such as RC 16 or CP 18, as to a reason the stimulation was halted or was returned to the previous setting(s). The patient or caregiver can then, for example, enter the reason or select from a set of reasons in a menu or the like or any combination thereof. In at least some embodiments, the programmer or another device stores the new setting(s) and the reason the new setting(s) was/were not acceptable. Such information may be provided on a future graph (see steps 404 and 406).

In optional step 414, after the limited period of time (if the stimulation has not been halted or returned to the previous setting(s)), a determination is made whether the adjusted stimulation is acceptable. For example, the programmer, such as RC 16 or CP 18, queries the patient or caregiver regarding the acceptability of the stimulation. If the stimulation is acceptable, then the method ends. In at least some other embodiments, optional steps 412 and 414 are repeated for one or more additional periods of time. For example, the patient or caregiver may be queried again about the acceptability of the adjusted stimulation at 30 minutes or 1, 2, 4, or 6 hours later (or any other suitable time) after the start of the stimulation and then queried a second time after 1, 2, 3, 5, or 7 days (or any other suitable time) after the start of the stimulation.

If the stimulation is not acceptable in step 414, then, in at least some embodiments, the process returns to step 408 (or step 404 or 406). In at least some other embodiments, in step 416, the stimulation system returns to the original stimulation setting(s). In at least some embodiments, the patient or caregiver is queried by the programmer, such as RC 16 or CP 18, whether to return to the original stimulation setting(s), as in step 416, or to provide one or more new settings, as in step 408 (or step 404 or 406). In at least some embodiment, the patient or caregiver is queried by the programmer, such as RC 16 or CP 18, as to a reason the new setting(s) was/were not acceptable. The patient or caregiver can then, for example, enter the reason or select from a set of reasons in a menu or the like or any combination thereof. In at least some embodiments, the programmer or another device stores the new setting(s) and the reason the new setting(s) was/were not acceptable. Such information may be provided on a future graph (see steps 404 and 406).

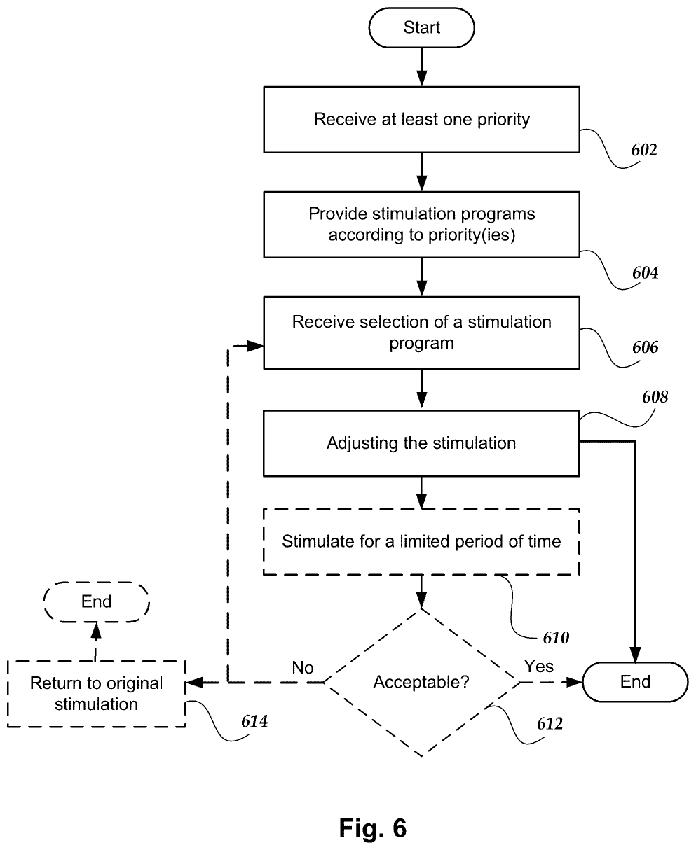

In at least some embodiments, a patient or caregiver can choose a priority (for example, a goal or objective) to improve stimulation. A priority may be used to improve the therapy or to address a decrease in the quality of the therapy. A patient or caregiver may use one or more priorities and may adjust priorities over time or may select different priorities for different times of day, activities (e.g., sleep, driving, walking, and so on), or the like or any other consideration. FIG. 6 illustrates one embodiment of a method for operation of an electrical stimulation system that includes an implantable control module configured for implantation in a patient and a lead coupled, or coupleable, to the implantable control module and having electrodes disposed along a distal portion of the lead.

In step 602, at least one priority is received. In at least some embodiments, a patient or clinician caregiver provides at least one priority to a programmer, such as RC 16 or CP 18. In at least some embodiments, each priority can be, for example, improvement in a symptom or a set of symptoms, a reduction in a side effect or a set of side effects, an improvement in a therapeutic effect or a set of therapeutic effects, avoidance of a side effect or set of side effects, a reduction in battery usage, or the like or any combination thereof. As an example, a patient may set priorities to improve tremor symptoms and avoid dyskinesia.

In at least some embodiments, a programmer, such as RC 16 or CP 18, includes an interface in which the patient or caregiver can input or select (for example, from a menu) one or more priorities or rank multiple priorities. In at least some embodiments, when multiple priorities are ranked, the programmer can produce a weighted score, based on the rankings, for particular setting(s) or modification(s) to setting(s).

In at least some embodiments, the patient or caregiver can also select one or more circumstances when the priority is to be effective such as, for example, a time of day, during a particular activity, when a particular symptom or side effect is detected, a time when medication is administered (which can also be adapted for alterations in scheduled medication), or the like or any combination thereof. The stimulation system can then be programmed to apply the modification(s) to the setting(s) associated with the priority. In at least some embodiments, the activity, symptom, or side effect may be detected by a sensor or other device or may be indicated by the user on the programmer. In at least some embodiments, the programmer can allow the patient or caregiver to select different priorities for different conditions (e.g., times of day, activities, side effects or symptoms, medication, or the like or any combination thereof).

In step 604, stimulation programs are provided by the system according to the priority/priorities. Any suitable method can be used to select stimulation programs including, but not limited to, selecting previously tested values of one or more stimulation parameters based on, for example, patient feedback, clinician feedback, sensed response to the stimulation, or the like or any combination thereof; selecting preprogrammed stimulation programs; obtaining stimulation programs from a database that may be accessed online or otherwise; determining stimulation programs using quantitative estimation methods (see, references cited above); known or estimated medication state; or the like or any combination thereof. The system can use any suitable method for selecting from among the available stimulation programs including, but not limited to, comparing the selected priority/priorities or rankings to similar information or rankings provided for the stimulation programs; estimating (see, references cited above) a response for individual stimulation programs and comparing with the selected priority/priorities or rankings; using an algorithm for selecting the stimulation programs (in at least some embodiments, the algorithm can be derived using machine learning techniques); or the like or any combination thereof. In at least some embodiments, the user can schedule a change in stimulation program to occur as a result of one or more predefined events or activities, or for a particular time of day or based on a medication schedule or the like or any combination thereof.

In step 606, a selection of a stimulation program is received. The selection can be made using any suitable user interface or device including, but not limited to, a touchscreen, a keyboard, a mouse, remote control, voice input, or the like or any combination thereof and may include selection from a menu, an input of the new setting, or the like or any combination thereof.

In step 608, the stimulation is adjusted according to the selection. In at least some embodiments, the programmer, such RC 16 or CP 18, send programming instructions to the IPG 14 or ETS 20. In at least some embodiments, the method ends at step 608. In at least some embodiments, the stimulation adjustment may require patient or caregiver approval, particularly, when the stimulation adjustment is not immediate (for example, when the stimulation adjustment is made at a particular time of day or for a particular activity). In at least some embodiments, the patient or caregiver may also designate the stimulation adjustment permanent or automatic (e.g., does not require patient or caregiver approval to initiate).

In at least some other embodiments, in optional step 610, the patient is stimulated for a limited period of time using the new setting (or settings). For example, the stimulation can be applied for 1, 5, 10, 15, 20, 30, 45, or 60 minutes; 1, 2, 4, 6, 8, 12, or 18 hours; or 1, 2, 5, or 7 days or more (or any other suitable time period). In at least some embodiments, the patient or caregiver can halt the stimulation at any time. In at least some embodiments, the patient or caregiver can return the stimulation to the previous setting(s) at any time. In at least some embodiment, the patient or caregiver is queried by the programmer, such as RC 16 or CP 18, as to a reason the stimulation was halted or was returned to the previous setting(s). The patient or caregiver can then, for example, enter the reason or select from a set of reasons in a menu or the like or any combination thereof. In at least some embodiments, the programmer or another device stores the new setting(s) and the reason the new setting(s) was/were not acceptable. Such information may be used in future determination of stimulation programs (see step 604).

In optional step 612, a determination is made whether the adjusted stimulation is acceptable. For example, the programmer, such as RC 16 or CP 18, queries the patient or caregiver regarding the acceptability of the stimulation. If the stimulation is acceptable, then the method ends. In at least some other embodiments, optional steps 610 and 612 are repeated for one or more additional periods of time. For example, the patient or caregiver may be queried again about the acceptability of the adjusted stimulation at 30 minutes or 1, 2, 4, or 6 hours later (or any other suitable time) after the start of the stimulation and then queried a third time after 1, 2, 3, 5, or 7 days (or any other suitable time) after the start of the stimulation.

If the stimulation is not acceptable, then, in at least some embodiments, the process returns to step 606 (or step 602 or 604). In at least some other embodiments, in step 614, the stimulation system returns to the original stimulation setting(s). In at least some embodiments, the patient or caregiver is queried by the programmer, such as RC 16 or CP 18, whether to return to the original stimulation setting(s), as in step 614, or to provide one or more new settings, as in step 606 (or step 602 or 604). In at least some embodiment, the patient or caregiver is queried by the programmer, such as RC 16 or CP 18, as to a reason the new setting(s) was/were not acceptable. In at least some embodiments, the programmer or another device stores the new setting(s) and the reason the new setting(s) was/were not acceptable. Such information may be used in future determination of stimulation programs (see step 604).

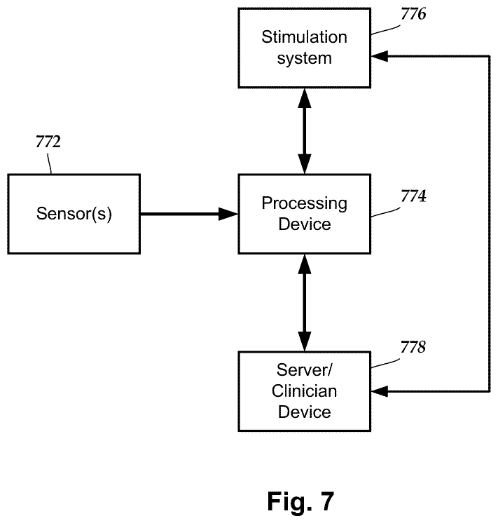

FIG. 7 illustrates one embodiment of a stimulation management system. The system includes one or more sensors 772, a processing device 774 for analyzing data from the sensor(s), a stimulation system 776 for providing stimulation therapy to a patient using the analyzed sensor data to adjust stimulation, and a server or clinician device 778 for storing the analyzed sensor data and communicating with the stimulation system.

Any suitable sensor(s) 772 can be used. Examples of suitable sensors include, but are not limited to, accelerometers, gyroscopes, GPS devices, pressure sensors, oximeters, inertial motion units, magnetometers, temperature sensors, glucose sensors, or the like or any combination thereof. A sensor can be wearable, implantable, or otherwise capable of receiving information about the patient. As an example, a sensor can be wearable or carried throughout a day or other period of time collecting data regarding the patient such, as for example, movement data to monitor movement disorders (for example, tremor, rigidity, gait disturbance, falling, dyskinesia, bradykinesia, or the like or any combination thereof).

The processing device 774 receives data from the sensor(s) 772 and processes the data to, for example, provide a score or other analysis of the data that is useful to monitor one or more symptoms, side effects, or therapeutic effects. Any suitable processing device can be used such as, for example, a dedicated processing device, a smartphone, tablet, computer, a stimulation system programmer (e.g., RC 16, CP 18, smartphone, tablet, computer, or the like), or the like or any combination thereof.

In at least some embodiments, each sensor 772 is coupled to the processing device 774. In at least some embodiments, the sensor 772 is physically attached to the processing device 774 or coupled to the processing device using a cable or the like to provide wired transmission between the processing device and the sensor. In at least some embodiments, the sensor 772 is wirelessly coupled, or coupleable, to the processing device 774. Wireless transmission may be continuous, periodic, or manually initiated.

In at least some embodiments, the processing device 774 calculates or otherwise determines one or more scores based on the data collected from the sensor(s). This can be a single cumulative score or an individualized score for each symptom, side effect, or therapeutic effect. For systems with multiple sensors, the scores can be cumulative scores for multiple sensors, individual scores for individual sensor, or any combination thereof. In at least some embodiments, the scoring algorithm can be modified, for example, via the server or clinician device 778.

The processing device 774 provides the score(s) to the stimulation system 776 (or an intermediate device). The score(s) can be used to modify or alter stimulation. For example, the processing device 774 can communicate with a stimulation system programmer (e.g., RC 16, CP 18, smartphone, tablet, computer, or the like) using a wired connection or wirelessly. As an example, a mobile device can be used as a stimulation system programmer and includes one or more applications to receive the score(s), modify or alter stimulation based on the score(s), and program the IPG 14. In at least some embodiments, the programmer can also interact with the patient or a caregiver and send them questions, tasks, education materials, or reports based on score(s) or sensor data. In at least some embodiments, the score(s) or sensor data can be displayed to the patient or caregiver.

The server or clinician device 778 receives information or data from one or more of the sensor(s) 772, the processing device 774, or the stimulation system 776. Examples of such information or data include, but are not limited to, sensor data, scores generated by the processing device, data or stimulation programs from the stimulation system, information (e.g., responses to queries, evaluations of stimulation, or the like) input by the patient or the caregiver, or the like or any combination thereof.

In at least some embodiments, there is a server 778 with a separate clinician device (for example, CP18). In at least some embodiments, the server 778 may include a portal (or separate portals) for one or more of the clinician (or other healthcare providers or persons associated with providing the stimulation therapy to the patient), manufacturer, caregiver, patient, or any other suitable person.

In at least some embodiments, the server or clinician device 778 can send data, analysis, stimulation programs, software updates, algorithm changes, or the like to one or more sensor(s) 772, the processing device 774, or the stimulation system 776. In at least some embodiments, the server or clinician device 778 may allow the clinician or another suitable individual to program the stimulation system remotely.

The stimulation system 776 provides stimulation therapy to the patient and includes, for example, the IPG 14 and other components illustrated in FIG. 1. In at least some embodiments, one or more of the sensors 772 can be part of the stimulation system. In at least some embodiments, data, program modifications, or the like can be stored on the processing device 774 or server/clinician device 776. In at least some embodiments, this information or portions of the information may be stored on the IPG 14.

In at least some embodiments, one or more of the processing device 774, the stimulation system 776, or the server/clinician device 776 can produce a stimulation report. The stimulation report may, or could, be provided to one or more of the patient, caregiver, clinician, or other suitable person. Examples of information that can be included in the stimulation report includes, but is not limited to, a summary of the patient's outcomes for the stimulation (which may be self-reported, determined from sensor or other measurements, or the like or any combination thereof); a summary of the patient's data from the sensor(s) 772 or the score(s) determined by the processing device 774; the stimulation parameters used at the time of data collection; medication records, schedules, and compliance; or the like or any combination thereof. In at least some embodiments, stimulation periods using different stimulation settings can be presented or summarized separately and comparisons can be presented for different stimulation settings. In at least some embodiments, the stimulation report can be provided using one or more different timescales including, but not limited to, hourly, daily, weekly, monthly, quarterly, semiannually, or yearly. In at least some embodiments, one or more of the patient, caregiver, clinician, or other suitable person can select what information is presented in the stimulation report, the format of the stimulation report, what information is accessible to others, or the like.

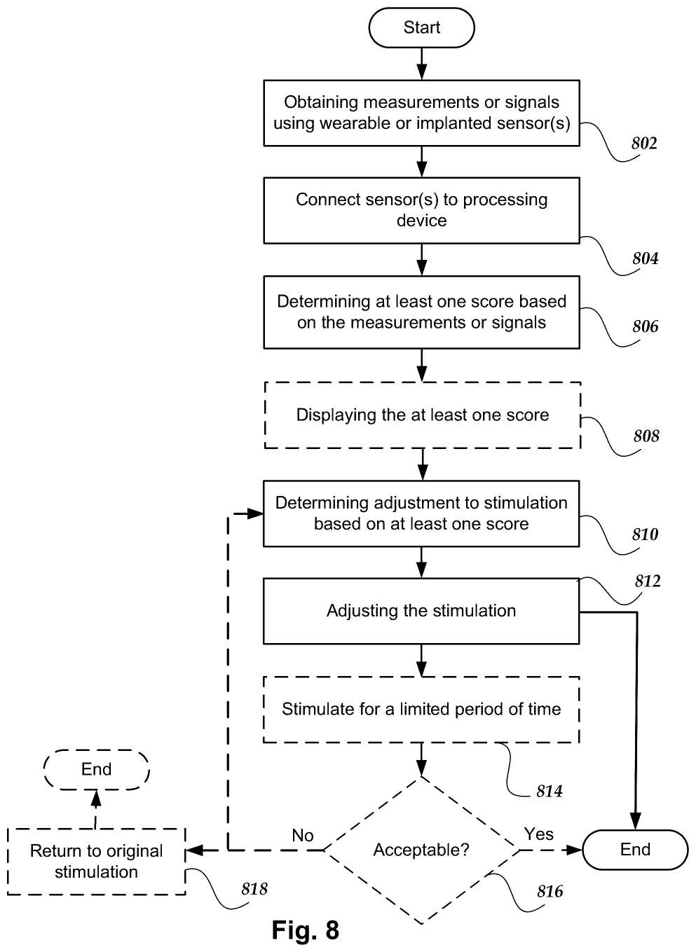

FIG. 8 illustrates one embodiment of a method for operation of a stimulation management arrangement. In step 802, sensor data is obtained using one or more sensors 772 (for example, one or more wearable or implanted sensor(s)). The sensor data can be, for example, sensor measurements or sensor signals. In step 804, the sensor(s) is/are connected to the processing device 774. In at least some embodiments, the connection is a physical connection (e.g., attachment or wired connection). In at least some embodiments, the connection is wireless. The connection may be continuous, periodic, irregular, or any other suitable arrangement.

In step 806, at least one score is determined by the processing device 774 based on the sensor data. Any suitable algorithm can be used to determine the score(s). As indicated above, the processing device may calculate a single cumulative score or an individualized score for each symptom, side effect, or therapeutic effect. For systems with multiple sensors, the scores can be cumulative scores for multiple sensors, individual scores for individual sensor, or any combination thereof.

In some embodiments, the system proceeds directly to either step 808 or step 810. In other embodiments, the system determines whether an adjustment to the stimulation is advised based on the score(s). Criteria may include, for example, whether it is likely that improvement to the score(s) can be made by adjustment of the stimulation, any priorities that the patient or caregiver has selected for stimulation, medication state, or the like or any combination thereof. If the system determines that adjustment is not advised, then the process ends. In yet other embodiments, the system may query a clinician or other medical professional whether an adjustment to the stimulation is advised. In either case, when the system determines that adjustment is not advised, then the process ends.

In at least some embodiments, in optional step 808, the score(s) is/are displayed by the processing device 774 or the stimulation system 776. In at least some embodiments, after the score(s) is/are displayed, the patient or caregiver is queried whether to adjust the stimulation. When the patient or caregiver consents to adjustment, the process continues. When the patient or caregiver does not consent, then the process ends.

In at least some embodiments, at step 808 or any subsequent step, the stimulation report described above can be provided to the patient, caregiver, clinician, or any other suitable individual. In at least some embodiments, the stimulation report is generated or presented automatically. In at least some embodiments, the stimulation report is generated or presented at the request of the patient, caregiver, clinician, or any other suitable individual.

In step 810, an adjustment to the stimulation is determined by the processing device 774 or the stimulation system 776 based on the score(s). Any suitable method can be used to determine an adjustment to the stimulation including, but not limited to, selecting previously tested values of one or more stimulation parameters based on, for example, patient feedback, clinician feedback, sensed response to the stimulation, or the like or any combination thereof; selecting preprogrammed stimulation programs; obtaining stimulation programs from a database that may be accessed online or otherwise; determining stimulation programs using quantitative estimation methods (see, references cited above); other patient's data; medication state; or the like or any combination thereof. The system can use any suitable method for selecting from among the available stimulation programs including, but not limited to, comparing the selected priority/priorities or rankings to similar information or rankings provided for the stimulation programs; estimating (see, references cited above) a response for individual stimulation programs and comparing with the selected priority/priorities or rankings; using an algorithm for selecting the stimulation programs (in at least some embodiments, the algorithm can be derived using machine learning techniques); comparing data recorded or estimated for similar medication states; accounting for the impact of a medication state; or the like or any combination thereof.

In at least some embodiments, the patient or caregiver is queried for confirmation that the determined adjustment to the stimulation is acceptable. If confirmation is not provided, then the process ends at step 810.

In step 812, the stimulation is adjusted or otherwise modified and provided to the patient. In at least some embodiments, the programmer, such RC 16 or CP 18, send programming instructions to the IPG 14 or ETS 20. In at least some embodiments, the method ends at step 812. In at least some embodiments, the patient or caregiver is queried for confirmation that the stimulation can be adjusted. If confirmation is not provided, then the process ends at step 810.

In at least some other embodiments, in optional step 814, the patient is stimulated for a limited period of time using the new setting (or settings). For example, the stimulation can be applied for 1, 5, 10, 15, 20, 30, 45, or 60 minutes; 1, 2, 4, 6, 8, 12, or 18 hours; or 1, 2, 5, or 7 days or more (or any other suitable time period). In at least some embodiments, the patient or caregiver can halt the stimulation at any time. In at least some embodiments, the patient or caregiver can return the stimulation to the previous setting(s) at any time. In at least some embodiment, the patient or caregiver is queried by the programmer, such as RC 16 or CP 18, as to a reason the stimulation was halted or was returned to the previous setting(s). The patient or caregiver can then, for example, enter the reason or select from a set of reasons in a menu or the like or any combination thereof. In at least some embodiments, the programmer or another device stores the new setting(s) and the reason the new setting(s) was/were not acceptable.

In optional step 816, after the limited period of time (if the stimulation has not been halted or returned to the previous setting(s)), a determination is made whether the adjusted stimulation is acceptable. For example, the programmer, such as RC 16 or CP 18, queries the patient or caregiver regarding the acceptability of the stimulation. If the stimulation is acceptable, then the method ends. In at least some other embodiments, optional steps 814 and 816 are repeated for one or more additional periods of time. For example, the patient or caregiver may be queried again about the acceptability of the adjusted stimulation at 30 minutes or 1, 2, 4, or 6 hours later (or any other suitable time) after the start of the stimulation and then queried a second time after 1, 2, 3, 5, or 7 days (or any other suitable time) after the start of the stimulation.

If the stimulation is not acceptable in step 816, then, in at least some embodiments, the process returns to step 810 (or step 802 or 806). In at least some other embodiments, in step 818, the stimulation system returns to the original stimulation setting(s). In at least some embodiments, the patient or caregiver is queried by the programmer, such as RC 16 or CP 18, whether to return to the original stimulation setting(s), as in step 818, or to provide one or more new settings, as in step 810 (or step 802 or 806). In at least some embodiment, the patient or caregiver is queried by the programmer, such as RC 16 or CP 18, as to a reason the new setting(s) was/were not acceptable. The patient or caregiver can then, for example, enter the reason or select from a set of reasons in a menu or the like or any combination thereof. In at least some embodiments, the programmer or another device stores the new setting(s) and the reason the new setting(s) was/were not acceptable.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each block of the flowcharts, and combinations of blocks in the flowcharts and methods disclosed herein, can be implemented by computer program instructions. These program instructions may be provided to a processor to produce a machine, such that the instructions, which execute on the processor, create means for implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. A method for operation of a stimulation system, the method comprising:
receiving a request to modify stimulation;
displaying a graph for at least one adjustable stimulation parameter;
indicating on the graph a current setting of each of the at least one adjustable stimulation parameter and a range around the current setting of at least one of the at least one adjustable stimulation parameter which represents at least one of a minor change in the stimulation based on a predefined minor change criteria or a moderate change in the stimulation based on a predefined moderate change criteria;
receiving a selection of a new setting for at least one of the at least one adjustable stimulation parameter; and
modifying the stimulation according to the selection.

2. The method of claim 1, wherein the indicating comprises indicating on the graph both the range representing the minor change and the range representing the moderate change.

3. The method of claim 1, wherein the indicating comprises indicating on the graph at least one previous setting for at least one of the at least one adjustable stimulation parameter.

4. The method of claim 3, wherein, for at least one of the at least one previous setting, the indication signifies an assessment of the stimulation provided by the previous setting.

5. The method of claim 3, wherein the indicating further comprises indicating on the graph an estimated disliked range based on a one of the at least one previous setting that has been assessed as providing disliked stimulation.

6. The method of claim 3, further comprising displaying a medication state for either the current setting or at least one of the at least one previous setting.

7. The method of claim 6, further comprising querying whether the modified stimulation is acceptable after providing the modified stimulation for a predefined period of time and continuing the modified stimulation when a response to the query indicates that the modified stimulation is acceptable.

8. The method of claim 7, further comprising halting the modified stimulation when the response to the query indicates that the modified stimulation is not acceptable.

9. The method of claim 7, further comprising returning to the previous stimulation when the response to the query indicates that the modified stimulation is not acceptable.

10. A method for operation of a stimulation system, the method comprising:
receiving, from a patient or a non-clinician caregiver, at least one priority for stimulation of the patient, wherein at least one of the at least one priority is selected from an improvement in a symptom or a set of symptoms, a reduction in a side effect or a set of side effects, an improvement in a therapeutic effect or a set of therapeutic effects, avoidance of a side effect or a set of side effects, a reduction in battery use, or any combination thereof;
identifying and providing at least one program based on the at least one priority;
receiving a selection of a one of the at least one stimulation program; and
stimulating the patient using the selected one of the at least one stimulation program.

11. The method of claim 10, wherein the receiving comprises ranking a plurality of the priorities.

12. The method of claim 10, wherein the identifying and providing comprises identifying and providing the at least one stimulation program based on the ranking of the plurality of priorities.

13. The method of claim 10, further comprising querying whether the selected one of the at least one stimulation program is acceptable after providing the selected one of the at least one stimulation program for a predefined period of time and continuing the selected one of the at least one stimulation program when a response to the query indicates that the selected one of the at least one stimulation program is acceptable.

14. The method of claim 13, further comprising, when the response to the query indicates that the selected one of the at least one stimulation program is not acceptable, either halting the selected one of the at least one stimulation program or returning to a previous stimulation program.

15. A method for operation of a stimulation management system, the method comprising:
obtaining, from at least one sensor, data for the patient regarding at least one symptom, at least one therapeutic effect, at least one side effect, or any combination thereof;
connecting the at least one sensor to a processing device;
delivering the data to the processing device;
processing the data to determine at least one score based on the data;
determining an adjustment to stimulation of the patient based on the at least one score; and
stimulating the patient according to the adjustment.

16. The method of claim 15, wherein the connecting comprises attaching, or coupling with a cable, at least one of the at least one sensor to the processing device.

17. The method of claim 15, further comprising displaying the at least one score for the patient or a non-clinician caregiver.

18. The method of claim 15, further comprising providing a stimulation report.

19. The method of claim 15, further comprising querying whether the adjustment to the stimulation is acceptable after providing the adjustment for a predefined period of time and continuing the adjustment when a response to the query indicates that the adjustment is acceptable.

20. The method of claim 19, further comprising, when the response to the query indicates that the adjustment is not acceptable, halting the stimulation or returning to a previous stimulation.
